(19) United States
(12) Patent Application Publication
(10) Pub. No.: US 2022/0211948 A1
(43) Pub. Date: Jul. 7, 2022

(54) SUPPLEMENTARY DEVICE FOR A MEDICAMENT DELIVERY DEVICE

(71) Applicant: SHL MEDICAL AG, ZUG (CH)

(72) Inventor: Chun Chu, Taipei (TW)

(21) Appl. No.: 17/611,857

(22) PCT Filed: Jun. 19, 2020
(86) PCT No.: PCT/EP2020/067100

§ 371 Date: Nov. 16, 2021

(30) Foreign Application Priority Data
Jul. 2, 2019 (EP) 19183775.6

(51) Int. Cl. A61M 5/315

(52) U.S. Cl. CPC A61M 5/3155; A61M 5/31581; A61M 2205/3327; A61M 2205/3306; A61M 5/31568

(57) ABSTRACT

A supplementary device configured to be mounted externally to a medicament delivery device provided with a dose setting knob, wherein the supplementary device has a sleeve configured to be arranged around a portion of the medicament delivery device, wherein the sleeve has a main body having a proximal sleeve end and a distal sleeve end, wherein the main body is provided with a flexible dose setting knob positioning structure which extend distally from the distal sleeve end.

### CROSS REFERENCE TO RELATED APPLICATIONS

The present application is a U.S. National Phase Application pursuant to 35 U.S.C. § 371 of International Application No. PCT/EP2020/067100 filed Jun. 19, 2020, which claims priority to European Patent Application No. 19183775.6 filed Jul. 2, 2019. The entire disclosure contents of these applications are herewith incorporated by reference into the present application.

### TECHNICAL FIELD

The present disclosure generally relates to medicament delivery devices, and in particular to a supplementary device for a medicament delivery device.

### BACKGROUND

Modern medicament delivery devices, such as auto-injectors, are designed to facilitate medicament administration in a manner which allows users to administer medicaments themselves. This freedom for patients to handle medicament delivery has led to the concept of adherence, or compliance, to become an increasingly important area in treatment of illnesses. Adherence involves monitoring of a patient's medication administration scheme as prescribed by a physician and evaluation of whether a prescribed medicament and medicament administration scheme has been successful or not in treating the illness of the patient.

It has been found that it is relatively common that a user does not administer the medication as prescribed. Reasons for a patient's failure to comply with the prescribed scheme include forgetfulness, pain associated with drug administration or discomfort experienced from side effects of the medication.

Failure to comply with a drug administration scheme may result in that the patient can experience poor recovery from an illness, and it may furthermore result in secondary diseases requiring additional medical attention. This may in turn bring unnecessary pressure on the healthcare system.

In view of the above, it is in the interest of both patients and the healthcare to facilitate monitoring to increase the occurrences of medicament administration as prescribed.

WO2007/107564 A1 discloses an electronic module for mechanical medication delivery devices and aims at monitoring the operation of a medication delivery device. This document discloses an electronic module that is attached onto a medication delivery device. The electronic module is capable of measuring acoustical and/or vibrational signals generated in response to relative movements of internal parts of the medication delivery device to which the electronic module is attached. Such internal parts can be mechanical parts which during movement generate for example acoustical sounds, such as click sounds. The electronic module is powered by a built-in battery which powers the module when for example a capacitive touch pad is activated. This activation is performed when for example a fingertip is positioned on the touch pad.

### SUMMARY

The electronic module disclosed in WO2007/107564 A1 can detect the click sounds in a high frequency range. One click corresponds to a certain amount of expelled medicament. By counting the number of clicks during an expelling sequence the amount of medicament expelled from the medicament delivery device can be calculated. The electronic module can however not determine the dose prior to medicament expulsion.

One way to detect the dose prior to medicament expulsion is by means of an optical sensor, such as the monitoring device disclosed in WO2018/158270.

One parameter that makes optical detection of the dose for medicament delivery devices such as medicament delivery devices of pen type difficult and imprecise is that the dose setting knob typically has a play relative to the housing. The play may be caused by manufacture tolerance, friction between components or wear and tear after long time of use and multiple manipulations. For example, some of the medicament delivery device comprises a thread engagement structure, such like the thread engagement between an outer housing of a delivery device and a dose setting knob or dose drum; or the dose setting knob/ dose drum and another coupling element, which is coupling to a driving assembly. Most of time, those devices with thread engagement using the pitch of the thread and the threading movement of the dose setting knob/dose drum achieved the dose setting mechanism. Ideally, the dose setting knob can only stopped at the start point of each pitches; however, the contact with the helical slope between two pitches creates friction, and how significant of the friction depended on multiple factors, such like the manufacture process, the chosen of material, times of use, force that end users applied during dose setting and administering; the friction might cause the dose setting knob stops at any point of the helical slope between two pitches under the unintentionally carrying/ transportation shaking of the medicament delivery device and also result as the play

This play can result in an incorrect determination of a set dose obtained by optical detection of the position of the dose setting knob. This is especially the case if the optical detection is arranged to track the movement of the dose setting knob or to measure the distance between the dose setting knob and the housing. The play may be so large that the dose setting knob could be in a position corresponding to zero dose units for the optical detection when in fact one dose unit has been set, because the maximum play away from the housing and the position of the dose setting knob when one unit has been set may be the same or essentially the same, especially when the detection is applied to a pen type injector with a large number of dose units, e.g. sixty units. Since the size of the injector usually will be small for easy handling and carrying, the gap or distance between each dose units will be also tiny.

An object of the present disclosure is to provide a supplementary device for a medicament delivery device which solves or at least mitigates problems of the prior art.

There is hence according to a first aspect of the present disclosure provided a supplementary device configured to be mounted externally to a medicament delivery device provided with a dose setting knob, wherein the supplementary device comprises: a sleeve configured to be arranged around a portion of the medicament delivery device, wherein the sleeve has a main body having a proximal sleeve end and a distal sleeve end, wherein the main body is provided with a flexible dose setting knob positioning structure which extend distally from the distal sleeve end.

A predetermined position of a dose setting knob of a medicament delivery device to which the supplementary device is attached, which has not been rotated to set a dose, defined by the axial extension of the dose setting knob positioning structure from the distal sleeve end may thereby be provided. The dose setting knob positioning structure may be configured to bias the dose setting knob towards a zero-dose position. A dose amount set by the dose setting knob may thereby be determined more precisely.

The dose setting knob positioning structure may be configured to bias the dose setting knob of a medicament delivery device to a default position. The default axial position may be a zero-dose position.

According to one embodiment the distal sleeve end is configured to be arranged closer to the dose setting knob than the proximal sleeve end when the supplementary device is mounted to the medicament delivery device.

According to one embodiment the dose setting knob positioning structure is flexible towards the distal sleeve end.

According to one embodiment the sleeve has a main body and the dose setting knob positioning structure is integral with the main body.

According to one embodiment the dose setting knob positioning structure comprises a first leg which extends in the distal direction from the distal sleeve end, wherein the first leg is angled relative to a longitudinal axis of the main body.

According to one embodiment the dose setting knob positioning structure comprises a second leg connected to the first leg, wherein the second leg extends in the distal direction from the distal sleeve end, wherein the second leg is angled relative to a longitudinal axis of the main body.

According to one embodiment the first leg and the second leg extend towards each other in the distal direction.

One embodiment comprises an electronics unit configured to be connected to the sleeve.

According to one embodiment the sleeve is hingedly connected to the electronics unit.

According to one embodiment the electronics unit comprises an optical sensor configured to detect an axial position of the dose setting knob.

According to one embodiment the dose setting knob positioning structure comprises a polymer material.

There is according to a second aspect of the present disclosure provided a medicament delivery device assembly comprising: a medicament delivery device comprising a housing and a dose setting knob configured to be moved relative to the housing, and a supplementary device according to the first aspect, configured to be mounted externally to the medicament delivery device.

The medicament delivery device may be a medicament delivery injection device such as a medicament delivery pen.

According to one embodiment the medicament delivery device has a central axis and the dose setting knob is configured to be moved along the central axis, wherein the dose setting knob positioning structure is configured to urge the dose setting knob in the distal direction to a predefined axial position.

According to one embodiment the dose setting knob positioning structure is configured to allow movement of the dose setting knob in the proximal direction.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the presently disclosed concept will now be described, by way of example, with reference to the accompanying drawings, in which:

FIG. 1 schematically show a side view of a play between the housing of a medicament delivery device and a dose setting knob;

FIG. 2 shows a perspective view of an example of a supplementary device for a medicament delivery device;

FIG. 3 shows a perspective view of the supplementary device in FIG. 2;

FIG. 4 shows a perspective view of the supplementary device in FIG. 2 attached to a medicament delivery device;

FIG. 5 is a perspective view of another example of a supplementary device;

FIG. 6 is a perspective view of the supplementary device in FIG. 5; and

FIG. 7 is a perspective view of the supplementary device in FIG. 5 attached to a medicament delivery device.

### DETAILED DESCRIPTION

The presently disclosed concept will now be described more fully hereinafter with reference to the accompanying drawings, in which exemplifying embodiments are shown. The presently disclosed concept may, however, be embodied in many different forms and should not be construed as limited to the embodiments set forth herein; rather, these embodiments are provided by way of example so that this disclosure will be thorough and complete, and will fully convey the scope of the presently disclosed concept to those skilled in the art. Like numbers refer to like elements throughout the description.

In the present disclosure, when the term “distal” is used, this refers to the direction pointing away from the dose delivery site. When the term “distal part/end” is used, this refers to the part/end of the supplementary device, or the parts/ends of the members thereof, which under use of the supplementary device when attached to a medicament delivery device is/are located furthest away from the dose delivery site. Correspondingly, when the term “proximal” is used, this refers to the direction pointing to the dose delivery site. When the term “proximal part/end” is used, this refers to the part/end of the supplementary device, or the parts/ends of the members thereof, which under use of the supplementary device is/are located closest to the dose delivery site.

Further, the term “longitudinal”, with or without “axis”, refers to a direction or an axis through the device or components thereof in the direction of the longest extension of the device and/or component.

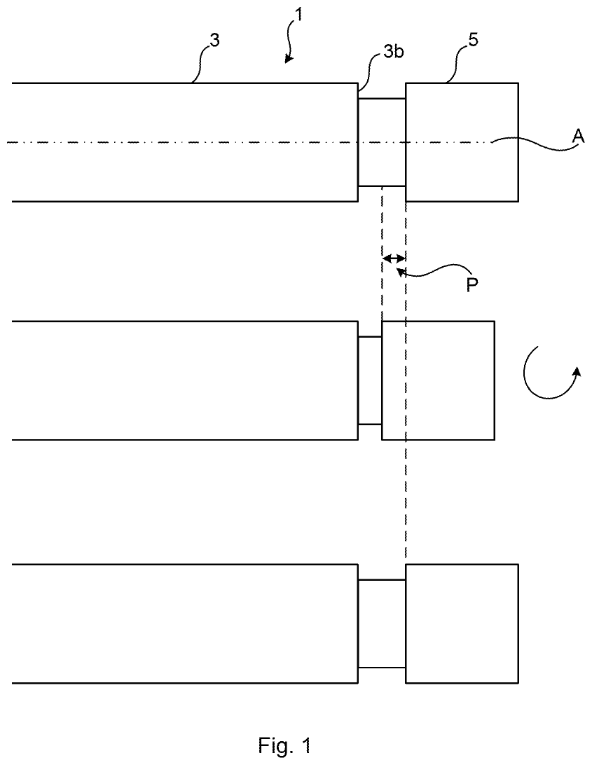

FIG. 1 schematically shows a side view of a general medicament delivery device 1 in three different states. The medicament delivery device 1 may for example be a medicament delivery pen.

The medicament delivery device 1 comprises a housing 3 and a dose setting knob 5. The dose setting knob 5 is configured to be rotated relative to the housing 3 to thereby move along a central axis A of the medicament delivery device 1. By rotating the dose setting knob 5 relative to the housing 3, the number of dose units to be expelled may be set. Typically, the dose setting knob 5 can be rotated in a first direction to increase the number of dose units and in a second direction opposite to the first direction to decrease the number of dose units.

The dose setting knob 5 is also able to move slightly linearly relative to the housing 3 with or without rotation. This may for example be due to manufacturing tolerances. The amount of such linear non-rotational movement is herein referred to as play P. For the medicament delivery device 1 in the top illustration of FIG. 1 the dose setting knob 5 is in a normal or default axial position relative to the housing 3. This default axial position is the zero-dose position, i.e. a position when the dose setting knob 5 is set to zero dose units. The dose setting knob 5 may be rotated relative to the housing 3 from the default axial position to set the number of dose units to be administered. When the dose unit or dose units have been set, the dose setting knob 5 is moved further away in the distal direction relative to a distal end 3b of the housing 3.

Due to the play P, the dose setting knob 5 may also be able to move closer to the distal end 3b of the housing 3 than in the default position, as shown in the middle illustration in FIG. 1. The dose setting knob 5 is in this case in a pushed in position.

A risk with the dose setting knob 5 being in the pushed in position is that the dose setting knob 5 may be rotated from the pushed in position in the first direction to set a dose. The amount that the dose setting knob 5 is moved in the distal direction when the dose setting knob 5 is being rotated from the pushed in position to set one dose unit may in some cases be the same or essentially the same as the play P as shown in the lowermost illustration in FIG. 1. The dose setting knob 5 has here been rotated an amount corresponding to one dose unit from the pushed in position. Hence, in such cases, rotation of the dose setting knob 5 to set one dose unit may result in that the dose setting knob 5 is set in the default axial position, i.e. in the zero-dose position. This may be problematic in case a supplementary device attached to the medicament delivery device 1 is employed to determine a dose as set by the position of the dose setting knob 5. As a result, the supplementary device may potentially not be able to precisely detect the number of doses as set by the dose setting knob 5. This may for example be the case if optical means are employed to determine the position of the dose setting knob 5. In the case shown in FIG. 1, an optical sensor would in the lowermost illustration consider the dose setting knob 5 to be in the default position whilst in fact a dose of one dose unit has been set.

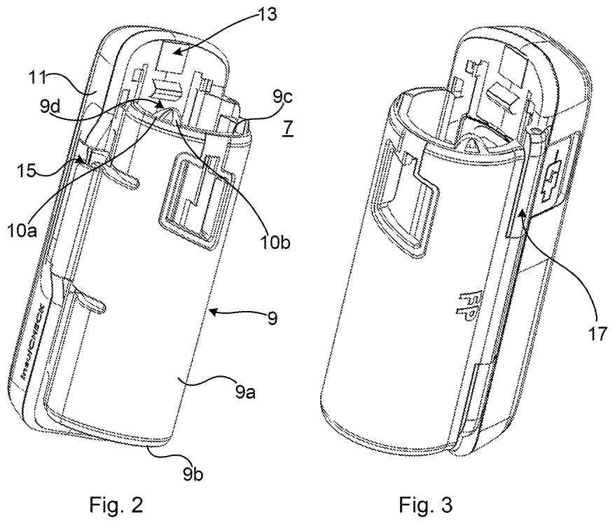

FIG. 2 shows an example of a supplementary device 7. The supplementary device 7 may also be referred to as an add-on device. The supplementary device 7 is configured to be mounted or attached externally to a medicament delivery device. Such a medicament delivery device may for example be a medicament delivery pen.

The supplementary device 7 comprises a sleeve 9. The supplementary device 7 comprises an electronics unit 11. The electronics unit 11 may form a supplementary device main body. The sleeve 9 may for example be hingedly connected to the electronics unit 11, as shown in FIG. 3. The sleeve 9 may be provided with hinges 17 configured to be attached to the electronics unit 11.

The sleeve 9 may be provided with a locking structure 15 configured to be releasably connected to the electronics unit 11. The locking structure 15 may for example have a snap-fit functionality to engage with the electronics unit 11, or it may be in the form of one or more magnets, or any other means for releasably locking two elements. The sleeve 9 may thereby be fixed to the electronics unit 11 in a position in which it encloses the housing of a medicament delivery device. The supplementary device 7 may hence be attached to a medicament delivery device in this manner.

The supplementary device 7 may comprise electronic components. The electronic components may be contained in the electronics unit 11. The electronics unit 11 may comprise an optical sensor 13. The optical sensor 13 may be configured to detect an axial position of a dose setting knob of a medicament delivery device. The electronics unit 11 may comprise processing circuitry connected to the optical sensor 13. The processing circuitry may be configured to determine a dose set by a dose setting knob based on the axial position of the dose setting knob as detected by the optical sensor 13.

The sleeve 9 has a main body 9a. The main body 9a has a proximal sleeve end 9b and a distal sleeve end 9c. The main body 9a has a flexible dose setting knob positioning structure 9d. The main body 9a may be provided with a plurality of dose setting knob positioning structures 9d distributed along the perimeter or edge of the distal sleeve end 9c. The main body 9a may for example be provided with exactly two dose setting knob positioning structures 9d distributed symmetrically relative to the longitudinal axis of the supplementary device 7.

The dose setting knob positioning structure 9d is mechanically flexible or elastic. The dose setting knob positioning structure 9d may be deformed or compressed towards the distal sleeve end 9c. The dose setting knob positioning structure 9d may be deformed or compressed when subjected to a force directed in the proximal direction. The dose setting knob positioning structure 9d in the present example is integral with the main body 9a. Alternatively, the dose setting knob positioning structure could be attached to the main body.

According to the example shown in FIGS. 2-4, the dose setting knob positioning structure 9d comprises a first leg 10a which extends in the distal direction from the distal sleeve end 9c. The first leg 10a is angled relative to the longitudinal axis of the main body 9a and of the supplementary device 7. The dose setting knob positioning structure 9d comprises a second leg 10b which extends in the distal direction from the distal sleeve end 9c. The second leg 10b is angled relative to the longitudinal axis of the main body 9a and of the supplementary device 7. The first leg 10a and the second leg 10b may extend towards each other in the distal direction. The first leg 10a and the second leg 10b may be tapering in the distal direction. The first leg 10a and the second leg 10b may form an arc or a curved structure, extending in the distal direction from the distal sleeve end 9c.

The dose setting knob positioning structure 9d may for example be made of a polymer material or a metal such as aluminium or stainless steel.

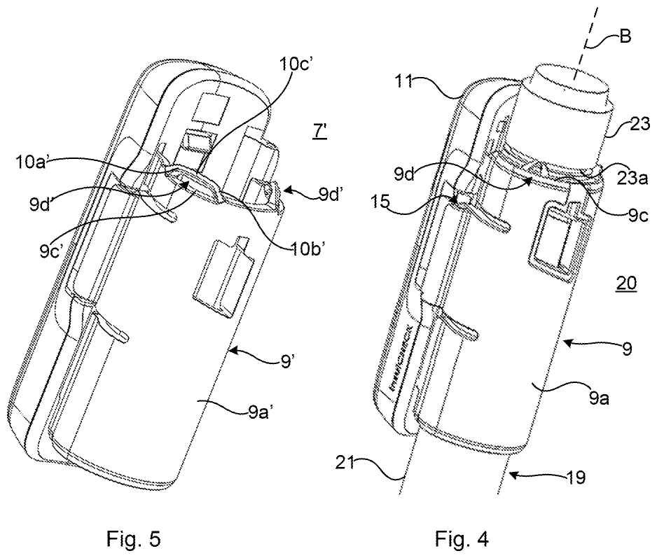

FIG. 4 shows the supplementary device 7 when fitted to a medicament delivery device 19. The medicament delivery device 19 has a housing 21 and a dose setting knob 23. The dose setting knob 23 is configured to be rotated relative to the housing 21 to set the dose to be administered. The dose setting knob 23 is thereby movable along a central axis B of the medicament delivery device 19, in particular relative to the housing 21. When a dose has been set, a user may administer the dose by pushing the dose setting knob 23 towards the housing 21.

The supplementary device 7 is attached to the medicament delivery device 19. The supplementary device 7 and the medicament delivery device 19 form a medicament delivery device assembly 20. The supplementary device 7 is attached to the housing 21 of the medicament delivery device 19. The sleeve 9 is hence arranged around the housing 21, and the locking structure 15 is connected to the electronics unit 11. The dose setting knob positioning structure 9d extends towards a proximal end 23a of the dose setting knob 23. The dose setting knob positioning structure 9d is dimensioned to extend from the distal sleeve end 9c such that the dose setting knob 23 is urged to a predefined axial position when no dose has been set. The predefined position is the default position. The dose setting knob positioning structure 9d hence urges or biases the dose setting knob 23 to the default position. Due to the elasticity, flexibility or resilience of the dose setting knob positioning structure 9d, the dose setting knob 23 may be moved towards the housing 3 from the default position when subjected to a large enough force, for example during medicament administration. The dose setting knob positioning structure 9d will however obtain its initial shape when this force subsides. The play P shown in FIG. 1 is hence normally not present and the dose setting knob 23 is therefore generally in the default position unless subjected to a compressive force towards the housing 3 or unless the dose setting knob 23 has been rotated to set a dose. The optical sensor 13 is thereby able to determine the axial position of the dose setting knob positioning structure 9d, resulting in a correct determination of any dose amount set by the dose setting knob 23.

FIG. 5 shows another example of a supplementary device 7′. The supplementary device 7′ is similar to the previously described supplementary device 7. According to this example, the sleeve 9′ is provided with a dose setting knob positioning structure 9d′ which has a first leg 10a′ and a second leg 10b' that form a broader curve than that of the previous example. The dose setting knob positioning structure 9d′ also has an intermediate section 10c′ connecting the first leg 10a′ and the second leg 10b′. The intermediate section 10c′ may have a central longitudinal axis that is parallel with or essentially parallel with the distal sleeve end 9c′. The main body 9a′ of the sleeve 9′ may be provided with a plurality of dose setting knob positioning structures 9d′ distributed along the perimeter or edge of the distal sleeve end 9c′. The main body 9a′ may for example be provided with exactly two dose setting knob positioning structures 9d′ distributed symmetrically relative to the longitudinal axis of the supplementary device 7′.

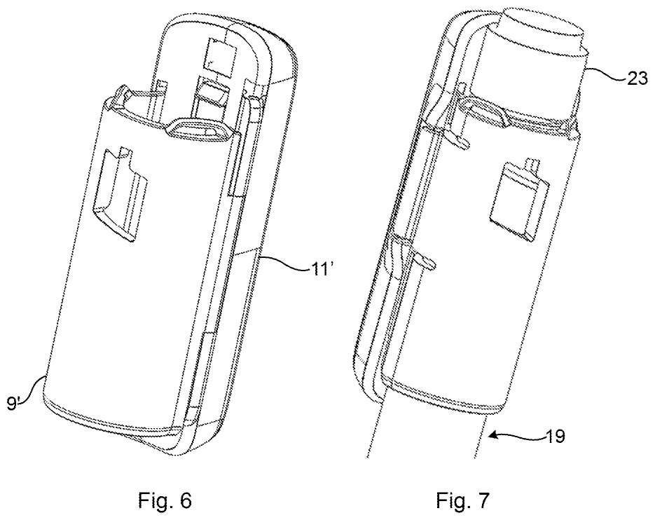

FIG. 6 depicts the supplementary device 7′ from another view, showing the hinged connection between the electronics unit 11′ and the sleeve 9′.

FIG. 7 shows the supplementary device 7′ fitted to the medicament delivery device 19. The dose setting knob positioning structure 9d′ biases the dose setting knob 23 towards the default position. The optical sensor of the supplementary device 7′ is thereby able to correctly determine a dose set by the dose setting knob 23.

The presently disclosed concept has mainly been described above with reference to a few examples. However, as is readily appreciated by a person skilled in the art, other embodiments than the ones disclosed above are equally possible within the scope of the presently disclosed concept, as defined by the appended claims.

1-14 (canceled)

15. A supplementary device configured to be mounted externally to a medicament delivery device provided with a dose setting knob, wherein the supplementary device comprises:
a sleeve configured to be arranged around a portion of the medicament delivery device,
wherein the sleeve has a main body having a proximal sleeve end and a distal sleeve end,
wherein the main body is provided with a flexible dose setting knob positioning structure which extend distally from the distal sleeve end.

16. The supplementary device as claimed in claim 15, wherein the distal sleeve end is configured to be arranged closer to the dose setting knob than the proximal sleeve end when the supplementary device is mounted to the medicament delivery device.

17. The supplementary device as claimed in claim 15, wherein the dose setting knob positioning structure is flexible towards the distal sleeve end.

18. The supplementary device as claimed in claim 15, wherein the sleeve has a main body and the dose setting knob positioning structure is integral with the main body.

19. The supplementary device as claimed in claim 15, wherein the dose setting knob positioning structure comprises a first leg which extends in the distal direction from the distal sleeve end, wherein the first leg is angled relative to a longitudinal axis of the main body.

20. The supplementary device as claimed in claim 18, wherein the dose setting knob positioning structure comprises a second leg connected to the first leg, wherein the second leg extends in the distal direction from the distal sleeve end, wherein the second leg is angled relative to a longitudinal axis of the main body.

21. The supplementary device as claimed in claim 20, wherein the first leg and the second leg extend towards each other in the distal direction.

22. The supplementary device as claimed in claim 15, comprising an electronics unit configured to be connected to the sleeve.

23. The supplementary device as claimed in claim 22, wherein the sleeve is hingedly connected to the electronics unit.

24. The supplementary device as claimed in claim 22, wherein the electronics unit comprises an optical sensor configured to detect an axial position of the dose setting knob.

25. The supplementary device as claimed in claim 15, wherein the dose setting knob positioning structure comprises a polymer material.

26. A medicament delivery device assembly comprising:
a medicament delivery device comprising a housing and a dose setting knob configured to be moved relative to the housing, and
a supplementary device as claimed in claim 1 configured to be mounted externally to the medicament delivery device.

27. The medicament delivery device assembly as claim in claim 26, wherein the medicament delivery device has a central axis and the dose setting knob is configured to be moved along the central axis, wherein the dose setting knob positioning structure is configured to urge the dose setting knob in the distal direction to a predefined axial position.

28. The medicament delivery device assembly as claimed in claim 27, wherein the dose setting knob positioning structure is configured to allow movement of the dose setting knob in the proximal direction.

29. An assembly comprising:
a medicament delivery device having a dose setting knob at a distal end of a housing; and
a supplementary device mounted externally to an outside surface of the distal end of the housing such that dose setting knob is accessible to a user, where the supplementary device comprises:
a main body comprising a sleeve; and
an electronics unit hingedly connected to the main body,
wherein the sleeve comprises a proximal sleeve end and a distal sleeve end, and
wherein the main body is provided with a positioning structure which extend distally from the distal sleeve end.

30. The assembly as claimed in claim 29, wherein the sleeve positioning structure is integral with the main body and the electronics unit is releasably connected to the main body.

31. The assembly as claimed in claim 29, wherein the positioning structure comprises a first leg which extends in the distal direction from the distal sleeve end, and wherein the first leg is angled relative to a longitudinal axis of the main body.

32. The assembly as claimed in claim 29, wherein the electronics unit comprises an optical sensor configured to detect an axial position of the dose setting knob.

33. The assembly as claimed in claim 29, wherein the dose setting knob moves along a central axis of the dose setting knob and wherein the positioning structure is configured to urge the dose setting knob in a distal direction to a predefined axial position.

34. The assembly as claimed in claim 29, wherein the dose setting knob moves axially in both a distal and proximal direction.
